(19) United States
(12) Patent Application Publication
(10) Pub. No.: US 2013/0012602 A1
(43) Pub. Date: Jan. 10, 2013

(54) METHODS OF USING ZNF365 GENETIC VARIANTS TO DIAGNOSE CROHN'S DISEASE

(75) Inventors: Talin Haritunians, Los Angeles, CA (US); Jerome I. Rotter, Los Angeles, CA (US); Kent D. Taylor, Ventura, CA (US); Stephan R. Targan, Santa Monica, CA (US)

(73) Assignee: CEDARS-SINAI MEDICAL CENTER, Los Angeles, CA (US)

(21) Appl. No.: 13/521,199

(22) PCT Filed: Jan. 13, 2011
(86) PCT No.: PCT/US11/21180

§ 371 Date: Jul. 9, 2012

Related U.S. Application Data
(60) Provisional application No. 61/294,635, filed on Jan. 13, 2010.

(51) Int. Cl. C12Q 1/68; G01N 27/447; A61P 1/00; C40B 30/04; A61K 35/00

(52) U.S. Cl. 514/789; 435/6.11; 506/9; 204/461

(57) ABSTRACT

The present invention relates to prognosing, diagnosing and treating of Crohn's disease. The invention also provides prognosis, diagnosis, and treatment that are based upon the presence of one or more genetic risk factors at the ZNF365 genetic locus

### CROSS-REFERENCE TO RELATED APPLICATIONS

The present application claims the benefit of the filing date of U.S. Provisional Application No. 61/294,635 filed Jan. 13, 2010, the disclosure of which is incorporated herein by reference in its entirety.

### FIELD OF INVENTION

The invention relates generally to the field of inflammatory disease, specifically to Crohn's disease.

### BACKGROUND

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (33-34). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (33). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (35). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (7, 35-38). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

In one embodiment, the invention provides a method of diagnosing susceptibility to Crohn's disease in an individual, comprising: obtaining a sample from the individual, assaying the sample to determine the presence or absence of a risk variant at the ZNF365 genetic locus, and diagnosing susceptibility to Crohn's disease in the individual based on the presence of the risk variant at the ZNF365 genetic locus. The risk variant can be selected from the group consisting of rs10740085, rs12768538, rs7068361, rs7071642, rs7076156, rs729739, rs10995271, rs12766391, rs10761659, and rs224120. Assaying of the sample comprises genotyping for one or more single nucleotide polymorphisms. The sample can be whole blood, plasma, serum, saliva, cheek swab, urine, or stool.

In another embodiment, the invention provides a method of determining a low probability of developing Crohn's disease in an individual, relative to a healthy subject, comprising: obtaining a sample from the individual, assaying the sample to determine the presence or absence of a protective variant at the ZNF365 genetic locus, and diagnosing a low probability of developing Crohn's disease in the individual, relative to a healthy subject, based upon the presence of the protective variant at the ZNF365 genetic locus. The risk variant can be selected from the group consisting of rs10740085, rs12768538, rs7068361, rs7071642, rs7076156, rs729739, rs10995271, rs12766391, rs10761659, and rs224120. Assaying of the sample comprises genotyping for one or more single nucleotide polymorphisms. The sample can be whole blood, plasma, serum, saliva, cheek swab, urine, or stool.

In a related embodiment, the invention provides a method of prognosing Crohn's disease in an individual, comprising: obtaining a sample from the individual, assaying the sample for the presence or absence of one or more genetic risk variants, and prognosing an aggressive form of Crohn's disease based on the presence of one or more risk variants at the ZNF365 genetic locus. The risk variant can be selected from the group consisting of rs10740085, rs12768538, rs7068361, rs7071642, rs7076156, rs729739, rs10995271, rs12766391, rs10761659, and rs224120. Assaying of the sample comprises genotyping for one or more single nucleotide polymorphisms. The sample can be whole blood, plasma, serum, saliva, cheek swab, urine, or stool.

In a further embodiment, the invention provides method of treating an individual for Crohn's disease, comprising: prognosing an aggressive form of Crohn's disease in the individual based on the presence of one or more risk variants at the ZNF365 genetic locus, and treating the individual, wherein the one or more risk variants are selected from rs10740085, rs12768538, rs7068361, rs7071642, rs7076156, rs729739, rs10995271, rs12766391, rs10761659, and rs224120. Assaying the sample comprises genotyping for one or more single nucleotide polymorphisms. The sample can be whole blood, plasma, serum, saliva, cheek swab, urine, or stool.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1. The genomic structure of the four isoforms of ZNF365 (A-D). Exon 4, unique to ZNF365D, harboring the associated SNP rs7076156 is also marked.

FIG. 2. Linkage disequilibrium and haplotype structure across the ZNF365 SNPs (generated in HAPLOVIEW). Region encompassing ZNF365 isoform D is noted. Top hits reported are marked with an asterisk (8, 10, 11). Rs7076156 is also marked, with rs7071642 immediately adjacent.

FIG. 3. Gel demonstrating expression of 379-bp ZNF365D was detected in ileum obtained from a CD patient undergoing small bowel surgery. ZNF365D expression is also observed in the adult kidney.

FIG. 4. Table 1 of ZNF365 SNPs associated with Crohn's Disease.

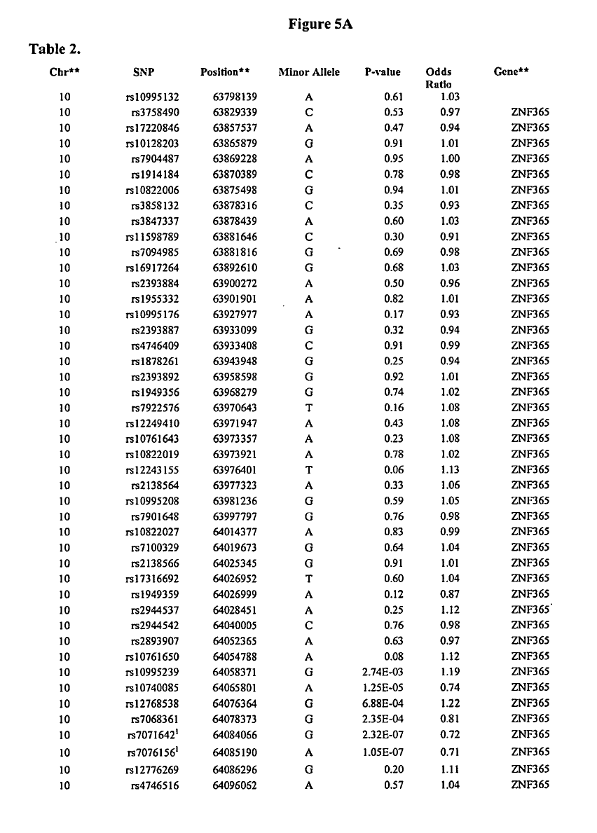

FIG. 5A-B. Table 2 of genotyped SNPs in 10q21.2.

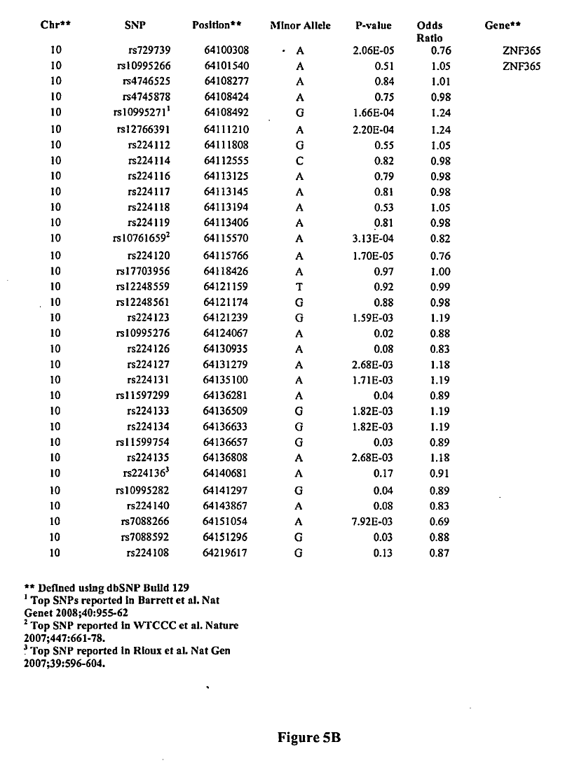

### DESCRIPTION OF THE INVENTION

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “inflammatory bowel disease” or “IBD” refers to gastrointestinal disorders including, but not limited to Crohn's disease (CD), ulcerative colitis (UC), and indeterminate colitis (IC). Inflammatory bowel diseases such as CD, UC, and IC are distinguished from all other disorders, syndromes, and abnormalities of the gastroenterological tract, including irritable bowel syndrome (IBS).

“Risk variant” as used herein refers to genetic variants, the presence of which correlates with an increase or decrease in susceptibility to Crohn's disease. Risk variants of Crohn's disease include, but are not limited to variants at the ZNF365 genetic locus, such as “haplotypes” and/or a set of single nucleotide polymorphisms (SNPs) on a gene or chromatid that are statistically associated. More preferably, risk variants can include, but are not limited to rs10740085, rs12768538, rs7068361, rs7071642, rs7076156, rs729739, rs10995271, rs12766391, rs10761659, and rs224120.

“Treatment” or “treating,” as used herein refer to both therapeutic treatment and prophylactic or preventative measures, wherein the object is to prevent, slow down and/or lessen the disease even if the treatment is ultimately unsuccessful. Those in need of treatment include those already with Crohn's disease as well as those prone to have Crohn's disease or those in whom Crohn's disease is to be prevented. For example, in Crohn's disease treatment, a therapeutic agent may directly decrease the pathology of IBD, or render the cells of the gastroenterological tract more susceptible to treatment by other therapeutic agents.

As used herein, “diagnose” or “diagnosis” refers to determining the nature or the identity of a condition or disease. A diagnosis may be accompanied by a determination as to the severity of the disease. Diagnosis as it relates to the present invention, relates to the diagnosis of Crohn's disease.

As used herein, “prognostic” or “prognosis” refers to predicting the probable course and outcome of IBD or the likelihood of recovery from IBD. The prognosis can include the presence, the outcome, or the aggressiveness of the disease.

As used herein, the term “biological sample” or “sample” means any biological material obtained from an individual from which nucleic acid molecules can be prepared. Examples of a biological sample include, but are not limited to whole blood, plasma, serum, saliva, cheek swab, urine, stool, or other bodily fluid or tissue that contains nucleic acid.

The inventors performed a genome-wide association study (GWAS) testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to 12, OmpC, and Cbir. The detection of protective and risk SNPs and/or haplotypes may be used to identify at risk individuals, predict disease course, and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, in the interest of identifying causal variants of Crohn's disease at 10q21, the inventors fine mapped the 10q21 region. The inventors genotyped 86 SNPs across the region of reported association (Chr. 10, position 63,798,139 to 64,219,617) in 1,683 CD cases and 1,049 non-IBD controls. Single marker and conditional analyses were performed using logistic regression (PLINK). ZNF365 isoform D expression was assessed using RT-PCR. Peak association with CD was observed within ZNF365 at rs7076156 and rs7071642, two SNPs in complete linkage disequilibrium (LD) (Table 1). Conditioning on nonsynonymous SNP rs7076156 (Ala62Thr) nullified all other significant associations and the threonine allele protected against CD (p=1.05×10⁷; OR 0.71; 23.6% in patients with CD and 30.1% in controls). Four isoforms of ZNF365 (A-D) have previously been identified and rs7076156 is located in an exon unique to ZNF365 isoform D. The inventors further detected expression of this isoform in a terminal ileum resection specimen from a patient with CD.

As further disclosed herein, the inventors demonstrate significant associations between CD and the ZNF365 locus. Conditional analyses show that a coding variant (rs7076156; Ala62Thr) confers protection against CD. Furthermore, mRNA for ZNF365 isoform D is expressed in small intestine. Taken together these data show that this variant explains the CD association observed at 10q21.

In one embodiment, the present invention provides a method of diagnosing a low probability of developing Crohn's Disease in an individual, relative to a healthy individual, by determining the presence or absence of one or more protective variants at the ZNF365 genetic locus, where the presence of the one or more protective variants at the ZNF365 genetic locus is indicative of a low probability of developing Crohn's Disease in an individual. In another embodiment, the one or more protective variants comprise rs10740085, rs12768538, rs7068361, rs7071642, rs7076156, rs729739, rs10995271, rs12766391, rs10761659, and/or rs224120.

In one embodiment, the present invention provides a method of diagnosing a risk of susceptibility to Crohn's Disease in an individual, relative to a healthy individual, by determining the presence or absence of one or more risk variants at the ZNF365 genetic locus, where the presence of the one or more risk variants at the ZNF365 genetic locus is indicative of susceptibility to Crohn's Disease in the individual. In another embodiment, the one or more risk variants comprise the SNP rs10740085, rs12768538, rs7068361, rs7071642, rs7076156, rs729739, rs10995271, rs12766391, rs10761659, and/or rs224120.

In one embodiment, the present invention provides a method of treating Crohn's Disease by determining the presence of a risk variant at the ZNF365 genetic locus and treating the individual. In another embodiment, the present invention provides a method of treating Crohn's Disease in an individual by determining the aberrant expression of ZNF365 and treating the individual. In another embodiment, the risk variant comprises the SNP rs10740085, rs12768538, rs7068361, rs7071642, rs7076156, rs729739, rs10995271, rs12766391, rs10761659, and/or rs224120.

In another embodiment, the present invention provides a method of prognosing Crohn's Disease by determining the presence or absence of one or more risk variants at the ZNF365 genetic locus and prognosing a complicated form of Crohn's Disease based on the presence of the one or more risk variants at the ZNF365 genetic locus.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (41).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VIC™ to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., (39). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (40, 45). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (41). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (42-43).

The technique of single strand conformational, polymorphism (SSCP) also may be used to detect the presence or absence of a SNP and/or a haplotype (44). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (45).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (46). Furthermore, one skilled in the art understands that, where the presence or absence of multiple alleles or haplotype(s) is to be determined, individual alleles can be detected by any combination of molecular methods (47). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

In the interest of identifying causal variants of Crohn's disease at 10q21, the inventors fine mapped the 10q21 region. The inventors genotyped 86 SNPs across the region of reported association (Chr. 10, position 63,798,139 to 64,219,617) in 1,683 CD cases and 1,049 non-IBD controls. Single marker and conditional analyses were performed using logistic regression (PLINK). ZNF365 isoform D expression was assessed using RT-PCR. Peak association with CD was observed within ZNF365 at rs7076156 and rs7071642, two SNPs in complete linkage disequilibrium (LD) (Table 1). Conditioning on nonsynonymous SNP rs7076156 (Ala62Thr) nullified all other significant associations and the threonine allele protected against CD (p=1.05×10⁻⁷; OR 0.71; 23.6% in patients with CD and 30.1% in controls). Four isoforms of ZNF365 (A-D) have previously been identified and rs7076156 is located in an exon unique to ZNF365 isoform D. The inventors further detected expression of this isoform in a terminal ileum resection specimen from a patient with CD.

As further disclosed herein, the inventors demonstrate significant associations between CD and the ZNF365 locus. Conditional analyses show that a coding variant (rs7076156; Ala62Thr) confers protection against CD. Furthermore, mRNA for ZNF365 isoform D is expressed in small intestine. Taken together these data show that this variant explains the CD association observed at 10q21.

### Example 2

A total of 1,683 predominantly Caucasian CD cases and 1,049 non-IBD controls were included in this analysis. CD subjects were recruited at Cedars-Sinai Medical Center Inflammatory Bowel Disease (CSMC IBD) Center and Wolfson Medical Center, Holon, Israel after diagnosis using standard clinical, endoscopic, and histological features (19). Controls, also of Caucasian descent, were recruited through the CSMC IBD Center (IBD patients' unrelated acquaintances and spouses of cases with no personal or family history of IBD or autoimmune disease); as part of the Pharmacogenetics and Risk of Cardiovascular disease (PARC) Study, a multicenter pharmacogenetic study of statin response (20-21); or from the National Laboratory for the Genetics of Israeli Populations at Tel-Aviv University; Tel-Aviv, Israel). All cases and controls provided informed consent prior to study participation and following approval of participating centers' institutional review boards.

### Example 3

The inventors applied a haplotype-tagging approach to the region previously associated with CD (chromosome 10, position 63,798,139 to 64,219,617) (8, 10-11) using Tagger as implemented in Haploview (22-23) and data from the International HapMap project, release 2. The inventors aimed to select SNPs compatible with the Illumina Infinium technology that tagged haplotypes with a frequency greater than 5% in the Caucasian population (24-25). Non-synonymous SNPs with a minor allele frequency in the Caucasian population >3% were also added to the initial genotyping panel of 86 SNPs. Genotyping for this study was performed as part of a project including a total of 7109 SNPs.

### Example 4

All genotyping was performed at the Medical Genetics Institute at Cedars-Sinai Medical Center using custom iSelect Infinium technology, following the manufacturer's protocol (Illumina, San Diego, Calif.) (26-27). Samples with genotyping success rates <98% or with gender discrepancies were excluded from analyses. The average genotyping rate of samples retained in the analysis was 99.9%. Twenty samples performed in duplicate yielded 100% concordance. SNPs were excluded if the test of Hardy-Weinberg equilibrium across the entire sample was p≦10⁻³; if the genotyping failure rate was >10%; if the minor allele frequency was <3%; or if the SNP had been selected for genotyping but was not found in the new dbSNP build at the time of analysis (dbSNP 129). These quality control steps left 78 SNPs in 10q21 for the analyses reported herein.

### Example 5

Single marker analysis for association with case/control status was performed using logistic regression (as implemented in PLINK v1.06) (28). Conditional logistic regression analysis was used to include allele load for the SNP being conditioned upon in the regression equation, and was performed using the condition function (PLINK).

### Example 6

Since ZNF365D has been reported to be expressed in kidney, commercially available total RNA extracted from human adult whole kidney tissue (Agilent Stratagene, La Jolla, Calif.) was used as a positive control for ZNF365D expression. Intestinal tissue was also collected from a Caucasian, non-smoking CD subject undergoing small bowel surgery at CSMC IBD Center for stricturing disease. There is a personal history of rheumatoid arthritis and a strong family history of autoimmune disease in this particular subject, and at the time of surgery the patient was being treated with anti-TNF medication (Humira). Tissue was stabilized for storage in RNAlater (Ambion, Austin, Tex.) and stored at room temperature until total RNA was extracted using the RiboPure Kit, following manufacturer's instructions (Ambion, Austin, Tex.). Because ZNF365D had been previously reported to have a short poly-A tail (GenBank NM_{—}199452.2), cDNA was synthesized from the total RNA template using random nonamers and the AffinityScript Multiple Temperature cDNA Synthesis kit (Agilent Stratagene, La Jolla, Calif.). The presence of the ZNF365D isoform was detected in a standard PCR reaction using the FailSafe PCR premix selection kit (Epicentre, Madison, Wis.). A single amplicon band at the expected size (379 bp) was seen with the FailSafe premix buffer H and ZNF365 isoform D specific PCR primers (Forward—5′ ATG TCT GCG CTG GGT CAG ATA 3′ and Reverse—5′ CTC CTG CAT AGG GAG GTG 3′ in exons 2 and 4, respectively; Invitrogen, Carlsbad, Calif.). PCR was preformed according to the following conditions: 10 min at 95°; followed by 40 cycles of: 30 sec at 95°; 1 min at 55°, 30 sec at 72°; and a final extension for 10 min at 72°.

### Example 7

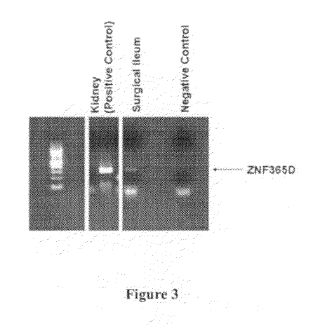

The inventors aimed to use a haplotype tagging approach to capture the major haplotypic variation in linkage disequilibrium with the 10q21 SNPs previous reported to be associated with CD (8, 10-11). Seventy-eight SNPs from this region were included in the final analysis of 1,683 CD cases and 1,049 non-IBD controls (Table 2). Ten SNPs exhibited highly significant associations (p<0.001) with the peak association observed at two SNPs, rs7076156 (OR=0.71; p=1.05×10⁻⁷) and rs7071642 (OR=0.72; p=2.32×10⁻⁷). These 2 SNPs were in complete linkage disequilibrium with each other (LD; r²=1.0) (Table 1 and FIG. 2). The inventors also confirmed association of CD with the previously reported SNPs rs10761659 (p=3.13×10⁻⁴) and rs10995271 (p=1.66×10⁻⁴) (10-11).

In order to determine whether the multiple associations were due to the high LD in this region and to identify the SNP or SNPs with the largest contribution to CD susceptibility, the inventors examined the effect of conditioning the CD association on each SNP in turn (Table 1). Conditioning on the most significantly associated SNP rs7076156 reduced all other CD associations to non-significance and regressed all odds ratios (OR) to 1 (with OR=1.2 for rs729739; Table 1). The regression of OR to 1, along with the change in P-values to become non-significant, demonstrate that the association observed between multiple SNPs in ZNF365 and CD is due to the LD between the associated markers within this region and what is potentially the causal variant, rs7076156 (Table 1). Analyses of the association between the haplotypes formed by the genotyped SNPs did not provide any further insight into the association between CD and this region beyond that of the association between CD and rs7076156.

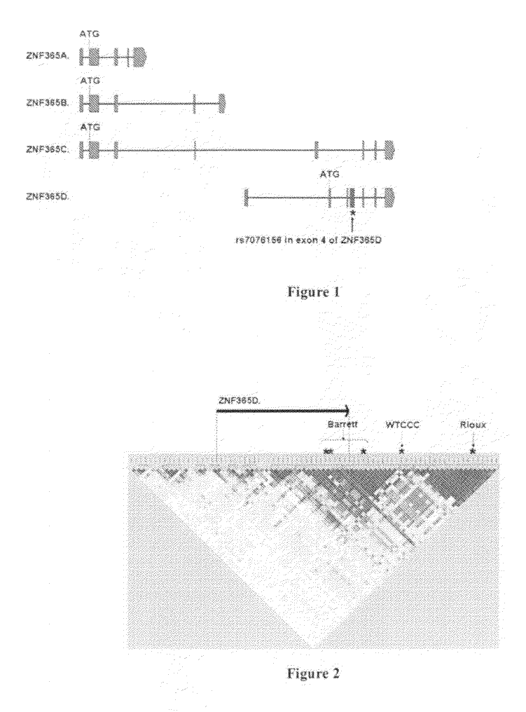

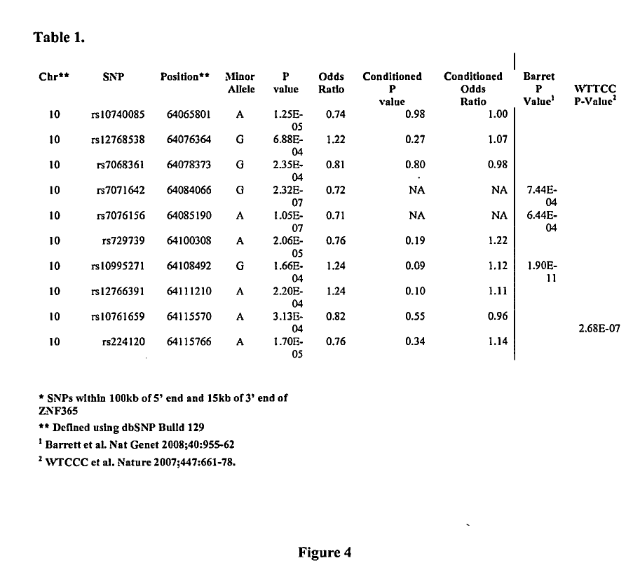

Four isoforms of ZNF365 (A-D) have been reported (FIG. 1) (14). rs7076156 is a nonsynonymous SNP (G>A; Ala62Thr) in exon 4 unique to ZNF365 isoform D. The minor allele (threonine allele) of Ala62Thr protected against CD (OR 0.71; Table 1) and had an allelic frequency of 23.6% in patients with CD and 30.1% in controls. In order to further elucidate a potential role for this functional variant in CD, the inventors focused attention on isoform D of ZNF365. RT-PCR was performed to evaluate the expression of ZNF365D in whole human kidney, a positive control tissue, and in human small intestine. The inventors confirmed previously reported expression of ZNF365D in the kidney (14) and detected expression of ZNF365D in cDNA from ileum obtained from a CD patient undergoing small bowel surgery (FIG. 3).

### Example 8

The inventors have characterized the association between CD and SNPs in the 10q21 region and have identified an association between a nonsynonymous Ala62Thr SNP located in the ZNF365D isoform (rs7076156, p=1.05×10⁻⁷; OR 0.71). Conditional analyses further demonstrated that this SNP accounts for the associations of other SNPs in the immediate region, including those in previous reports and confirmed in this study (Table 1; rs10761659, p=3.13×10⁻⁴; rs10995271, p=1.66×10⁻⁴) (10-11, 29). Thus, even though the LD between the ZNF365D Ala62Thr variant and the SNPs in some previous reports was low (r² between Ala62Thr rs7076156 and rs10995271 is 0.19 and rs10761659 is 0.37), this conditional analysis shows that the ZNF365D Ala62Thr variant accounts for the association observed in these reports. Since expression of the ZNF365D isoform has thus far not been reported in intestine, the inventors tested for and subsequently observed the expression of this isoform in human intestine from a CD patient undergoing surgery for strictures (FIG. 3). When taken together, these observations support expression of the ZNF365D isoform with the Threonine allele in human intestine is associated with CD.

In summary, the inventors provide evidence from both a genetic and expression perspective that ZNF365 is a convincing candidate gene for CD susceptibility, having demonstrated an association with a coding variant rs7076156 that confers strong protection against CD. Conditional analysis indicated the causal variant in the region is likely to be this nonsynonymous SNP that is located in an exon unique to one of four isoforms of this gene. Finally, the inventors have demonstrated expression in the ileum of a CD subject. When taken together, these observations point to this SNP as a causal variant for CD within the 10q21 region.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

### REFERENCES

1. Shih, et al., World J Gastroenterol 2008; 14:390-400.
2. Van Limbergen, et al.,. Am J Gastroenterol 2007; 102:2820-31.
3. Cho, J H. Nat Rev Immunol 2008; 8:458-66.
4. Duerr, et al., Science 2006; 314:1461-3.
5. Yamazaki, et al., Hum Mol Genet 2005; 14:3499-506.
6. Hugot, et al., Nature 2001; 411:599-603.
7. Ogura, et al., Nature 2001; 411:603-6.
8. Rioux et al., Nat Genet 2007; 39:596-604.
9. Hampe, et al., Nat Genet 2007; 39:207-11.
10. Consortium WTCC. Genome-wide association study of 14,000 cases of seven common diseases and 3,000 shared controls. Nature 2007; 447:661-78.
11. Barrett, et al., Nat Genet 2008; 40:955-62.
12. Safford, et al., Nat Immunol 2005; 6:472-80.
13. Nagase, et al., DNA Research 1998; 5:355-364.
14. Gianfrancesco, et al., Am J Hum Genet 2003; 72:1479-91.
15. Gianfrancesco, et al., Gene 2004; 339:131-8.
16. Wang, et al., Proc Natl Acad Sci USA 2006; 103:6512-7.
17. Hirohashi, et al., Oncogene 2006; 25:6048-55.
18. Ombra, et al., Am J Hum Genet 2001; 68:1119-29.
19. Mow, et al., Gastroenterology 2004; 126:414-24.
20. Simon, et al., Am J Cardiol 2006; 97:843-50.
21. Krauss, et al., Circulation 2008; 117:1537-44.
22. De Bakker P I. Tagger broad-mit 2004.
23. Barrett, et al., Bioinformatics 2005; 21:263-5.
24. Consortium I H. The International HapMap Project. Nature 2003; 426:789-796.
25. Frazer, et al., Nature 2007; 449:851-61.
26. Gunderson, et al., Pharmacogenomics 2006; 7:641-8.
27. Gunderson, K L, et al., Methods Enzymol 2006; 410:359-76.
28. Purcell, et al., Am J Hum Genet 2007; 81:559-75.
29. Franke, et al., Nat Genet 2008; 40:713-5.
30. Glas, et al., Am J Gastroenterol 2009; 104:665-72.
31. Kent, et al., Genome Res 2002; 12:996-1006.
32. Karolchik, et al., Nucleic Acids Res 2003; 31:51-4.
33. Podolsky, et al., N Engl J Med 347, 417 (2002).
34. Loftus, et al., Gastroenterology 126, 1504 (2004).
35. Vermeire, et al., Genes Immun 6, 637 (2005).
36. Hugot, et al., Nature 411, 599 (2001).
37. Rioux, et al., Nat Genet 29, 223 (2001).
38. Peltekova, et al., Nat Genet 36, 471 (2004).
39. Jarcho et al. in Dracopoli et al., Current Protocols in Human Genetics pages 2.7.1-2.7.5, John Wiley & Sons, New York.
40. Kutyavin, et al., Nucleic Acids Research 28:655-661 (2000).
41. Mullis, et al. (Eds.), The Polymerase Chain Reaction, Birkhauser, Boston, (1994).
42. Delwart, et al., Science 262:1257-1261 (1993).
43. White, et al., Genomics 12:301-306 (1992).
44. Hayashi, K., Methods Applic. 1:34-38 (1991).
45. Innis, et al., (Ed.), PCR Protocols, San Diego: Academic Press, Inc. (1990).
46. Winter, et al., Proc. Natl. Acad. Sci. 82:7575-7579 (1985).
47. Birren, et al. (Eds.) Genome Analysis: A Laboratory Manual Volume 1 (Analyzing DNA) New York, Cold Spring Harbor Laboratory Press (1997).

1. A method of diagnosing susceptibility to Crohn's disease in an individual, comprising:
obtaining a sample from the individual;
assaying the sample to determine the presence or absence of a risk variant at the ZNF365 genetic locus; and
diagnosing susceptibility to Crohn's disease in the individual based on the presence of the risk variant at the ZNF365 genetic locus.

2. The method according to claim 1, wherein the risk variant is selected from the group consisting of rs10740085, rs12768538, rs7068361, rs7071642, rs7076156, rs729739, rs10995271, rs12766391, rs10761659, and rs224120.

3. The method according to claim 1, wherein the risk variant is rs7076156.

4. The method according to claim 1, wherein the risk variant is rs7071642.

5. The method of claim 1, wherein assaying the sample comprises genotyping for one or more single nucleotide polymorphisms.

6. The method according to claim 1, wherein the sample is whole blood, plasma, serum, saliva, cheek swab, urine, or stool.

7. A method of determining a low probability of developing Crohn's disease in an individual, relative to a healthy subject, comprising:
obtaining a sample from the individual;
assaying the sample to determine the presence or absence of a protective variant at the ZNF365 genetic locus; and
diagnosing a low probability of developing Crohn's disease in the individual, relative to a healthy subject, based upon the presence of the protective variant at the ZNF365 genetic locus.

8. The method according to claim 7, wherein the protective variant is selected from the group consisting of rs10740085, rs12768538, rs7068361, rs7071642, rs7076156, rs729739, rs10995271, rs12766391, rs10761659, and rs224120.

9. The method according to claim 7, wherein the protective variant is rs7076156.

10. The method according to claim 7, wherein the protective variant is rs7071642.

11. The method of claim 7, wherein assaying the sample comprises genotyping for one or more single nucleotide polymorphisms.

12. The method according to claim 7, wherein the sample is whole blood, plasma, serum, saliva, cheek swab, urine, or stool.

13. A method of prognosing Crohn's disease in an individual, comprising:
obtaining a sample from the individual;
assaying the sample for the presence or absence of one or more genetic risk variants; and
prognosing an aggressive form of Crohn's disease based on the presence of one or more risk variants at the ZNF365 genetic locus.

14. The method according to claim 13, wherein the risk variant is selected from the group consisting of rs10740085, rs12768538, rs7068361, rs7071642, rs7076156, rs729739, rs10995271, rs12766391, rs10761659, and rs224120.

15. The method of claim 13, wherein assaying the sample comprises genotyping for one or more single nucleotide polymorphisms.

16. The method according to claim 13, wherein the sample is whole blood, plasma, serum, saliva, cheek swab, urine, or stool.

17. A method of treating an individual for Crohn's disease, comprising:
prognosing an aggressive form of Crohn's disease in the individual based on the presence of one or more risk variants at the ZNF365 genetic locus; and
treating the individual,
wherein the one or more risk variants are selected from rs10740085, rs12768538, rs7068361, rs7071642, rs7076156, rs729739, rs10995271, rs12766391, rs10761659, and rs224120.

18. The method of claim 17, wherein assaying the sample comprises genotyping for one or more single nucleotide polymorphisms.

19. The method according to claim 17, wherein the sample is whole blood, plasma, serum, saliva, cheek swab, urine, or stool.
